(19) United States
(12) Patent Application Publication
(10) Pub. No.: US 2022/0262500 A1
(43) Pub. Date: Aug. 18, 2022

(54) MEDICAL LOGISTIC PLANNING TOOL FOR CHEMICAL, BIOLOGICAL, RADIOLOGICAL, AND NUCLEAR CASUALTY ESTIMATION

(71) Applicant: The United States of America as Represented by the Secretary of the Navy, Silver Spring, MD (US)

(72) Inventors: Karl M. Matlage, Fairfax, VA (US); Andrew Olson, San Diego, CA (US); Tracy Negus, San Diego, CA (US); Vern Wing, Coronado, CA (US); Patrick Givens, Huntsville, AL (US); Josh Harper, Harvest, AL (US); James Young, Huntsville, AL (US); Jennifer Shah, Madison, AL (US)

(73) Assignee: The United States of America as Represented by the Secretary of the Navy, Silver Spring, MD (US)

(21) Appl. No.: 17/516,473

(22) Filed: Nov. 1, 2021

Related U.S. Application Data
(63) Continuation-in-part of application No. 15/004,022, filed on Jan. 22, 2016, now abandoned.
Application No. 15/004,022 is a continuation-in-part of application No. 14/192,521, filed on Feb. 27, 2014, now Pat. No. 10,706,129.
(60) Provisional application No. 63/107,710, filed on Oct. 30, 2020.
(60) Provisional application No. 62/107,077, filed on Jan. 23, 2015.
(60) Provisional application No. 61/769,805, filed on Feb. 27, 2013.

(51) Int. Cl. G16H 40/20; G16H 70/60; G16H 50/30; G06Q 50/26

(52) U.S. Cl. CPC G16H 40/20; G06Q 50/26; G16H 50/30; G16H 70/60

(57) ABSTRACT

The present invention discloses a computer implemented method for planning a medical logistic support for an operational scenario involving a CBRN event, with implementable plan include medical network laydowns, medical facility, equipment, personnel and medical consumable requirements. The method allow user to generate medical laydowns within a theater, model patient care, and identify the demand for medical care, model casualty transportation requirements, project patient clinical outcomes from the Point of Injury (POI) through evacuation from theater.

### CROSS-REFERENCE TO RELATED APPLICATIONS

This application claims priority to U.S. provisional application 63/107,710 filed Oct. 30, 2020 and is a continuation-in-part application of Patent application Ser. No. 15/004,022 filed on Jan. 22, 2016, which claims priority to 62/107,072 filed Jan. 23, 2015, and is a continuation-in-part application of Patent application Ser. No. of 14/192,521 filed on Feb. 27, 2014 (Pat. No. 10,706,129, issued on Jul. 7, 2020), which claims priority to Provisional Application No. 61/769,805 filed on Feb. 27, 2013, which are all hereby incorporated herein by reference in their entireties.

### STATEMENT REGARDING FEDERALLY SPONSORED RESEARCH OR DEVELOPMENT

This invention was made with Government support under contracts W911QY-11-D-0058, N62645-12-C-4076 and N62645-20-D-5008 that were awarded by the OSD DHA, OPNAV (N81), and the Joint Staff. The Government has certain rights in the invention.

### FIELD OF THE INVENTION

The present invention relates in general to a computer tool used in medical logistics planning and modeling. More particularly, this invention relates to computer implemented programs, and method to build medical logistic plans for chemical, biological, radiological, or nuclear (CBRN) casualties and to plan and evaluate treatment network and supply requirements for treating these casualties.

### BACKGROUND

In its earlier efforts, the Department of Defense (DoD) have developed two accredited medical logistic tools: the Medical Planner's Toolkit (MPTK) and the Joint Medical Planning Tool (JMPT), which are currently used by the medical planning community, to develop casualty estimates, estimate requirements for theater hospitalization and [symbol] VIII supply, conduct risk assessments, and determine the medical treatment network that would optimally treat the expected patient stream for conventional casualties. However, there is a need to develop tools to estimate chemical, biological, radiological, and nuclear (CBRN) casualties and plan treatment network and supply requirements for treating these casualties. To meet this requirement the current computer tools must be modified to include chemical, biological, radiological, and nuclear patient conditions, treatment profiles, supplies, and other associated data.

### DETAILED DESCRIPTION OF DRAWINGS

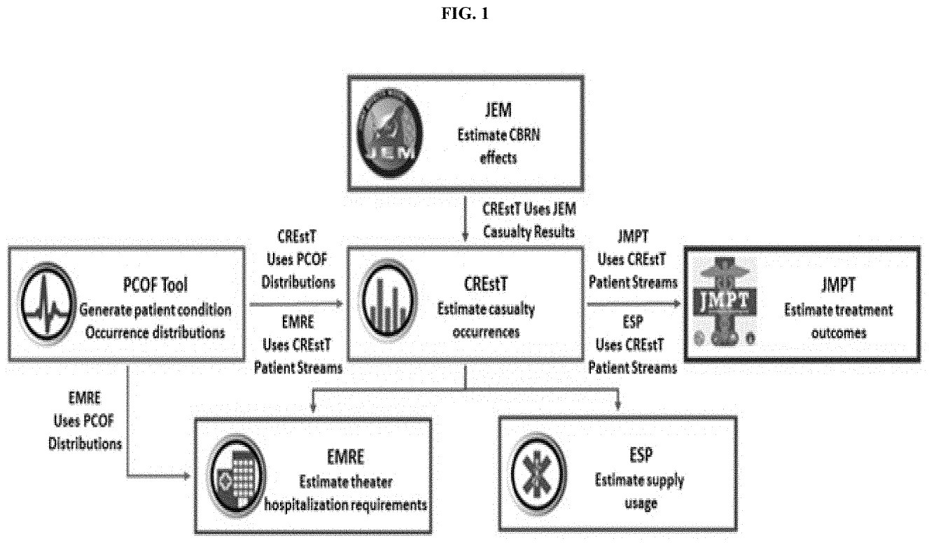

FIG. 1 Flow chart showing integration of JEM, MPTk and JMPT software for modeling and simulation of CBRN casualties.

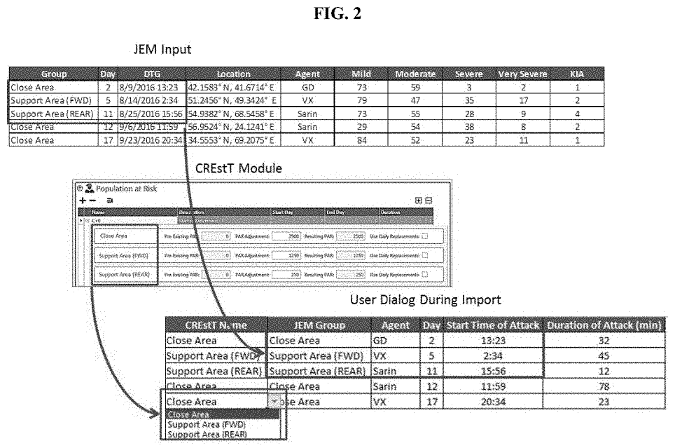

FIG. 2 shows an example of a potential CREstT user dialog.

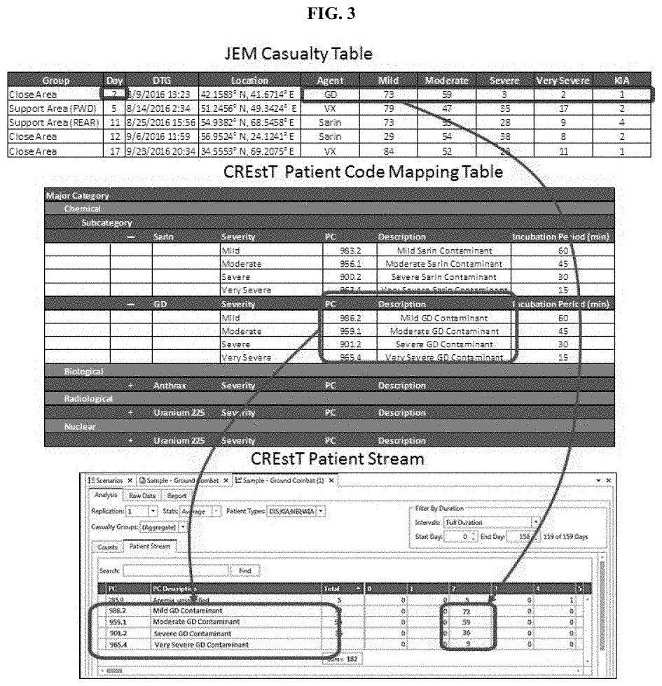

FIG. 3 represents an example of the process flow for integrating the CBRN casualty estimate from JEM with the conventional patient stream in CREstT.

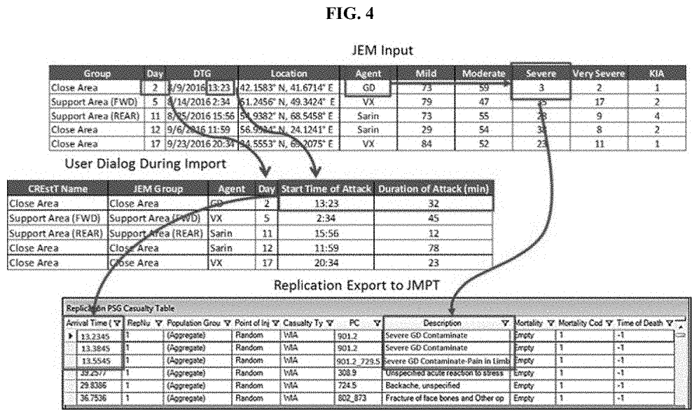

FIG. 4 shows how to export a CREstT Patient Stream

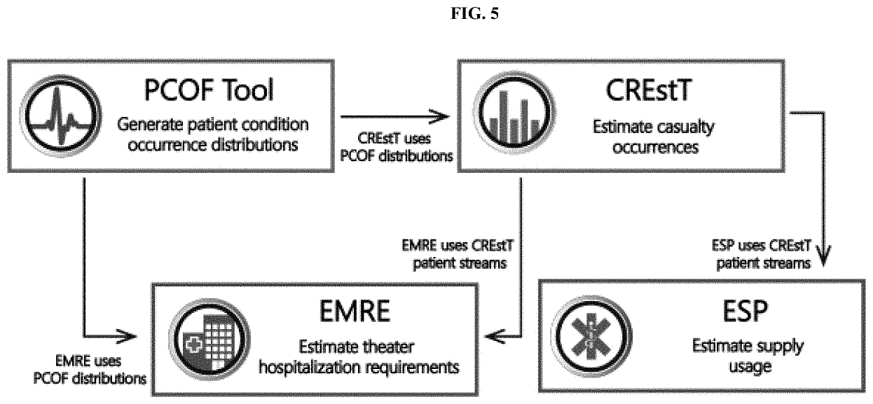

FIG. 5 flow chart of different toolbox of a MPTk software

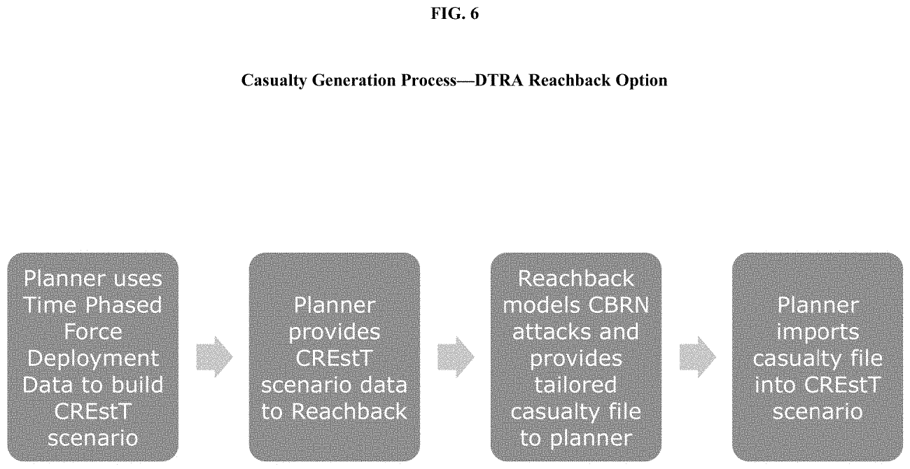

FIG. 6 shows the process of casualty generation using DTRA reachback option

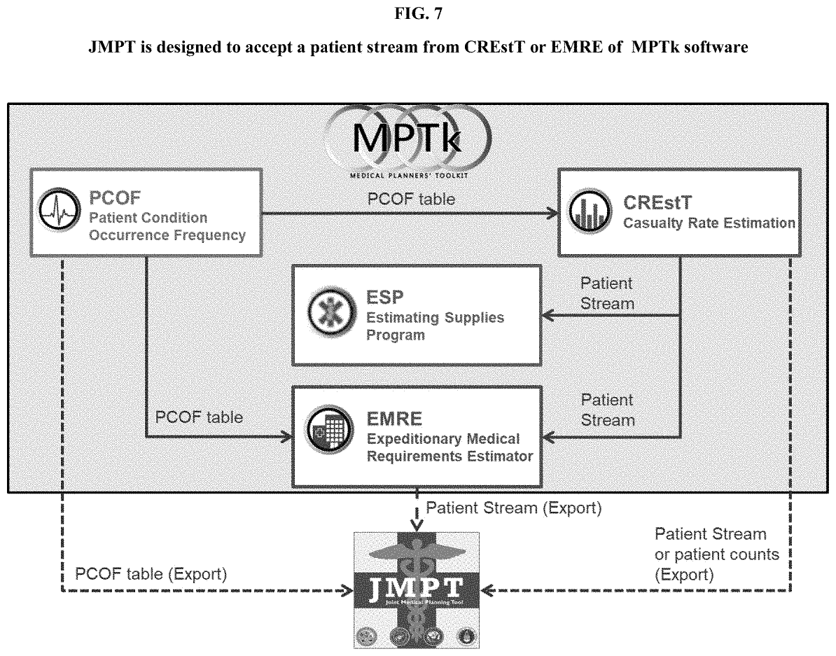

FIG. 7 shows the how JMPT is integrated with MPTk software

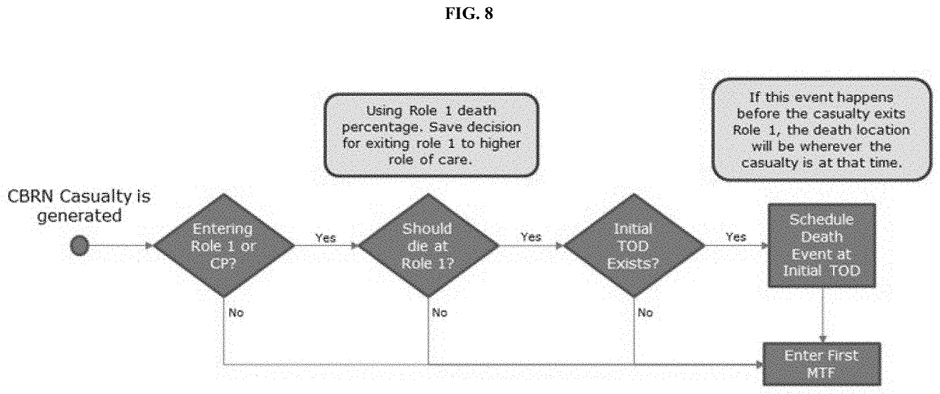

FIG. 8 shows role 1 chemical casualty mortality algorithms

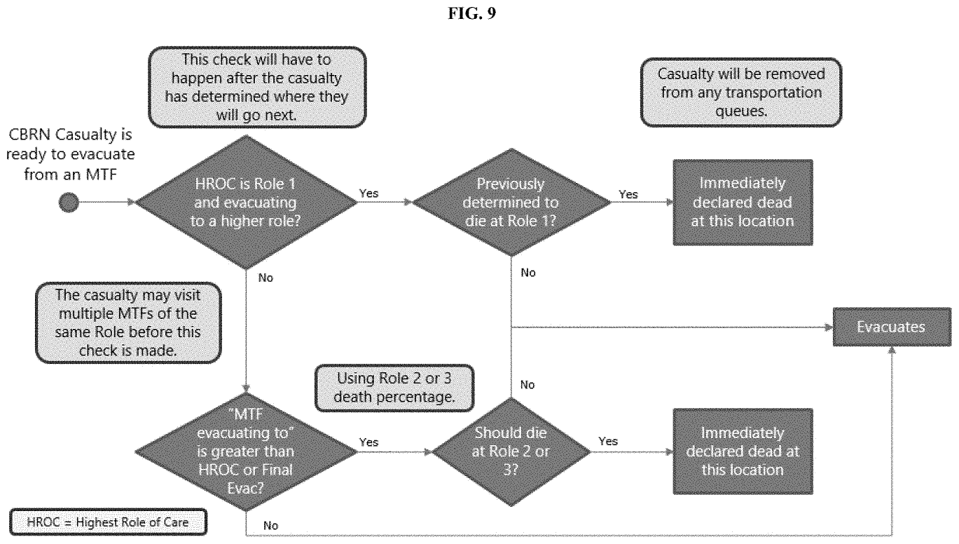

FIG. 9 shows higher role of care chemical casualty mortality algorithms

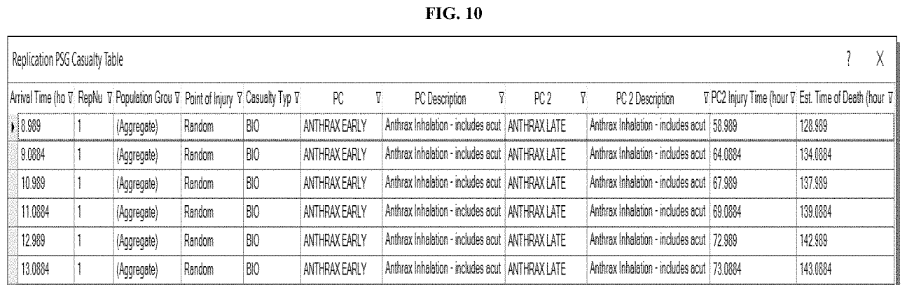

FIG. 10 shows an example of a PSG with biological casualties.

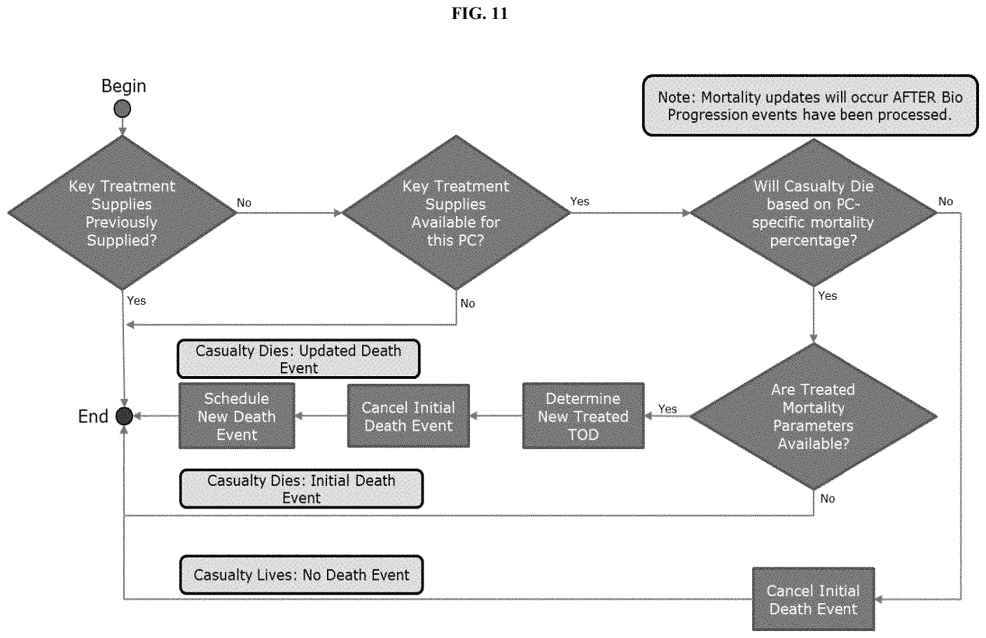

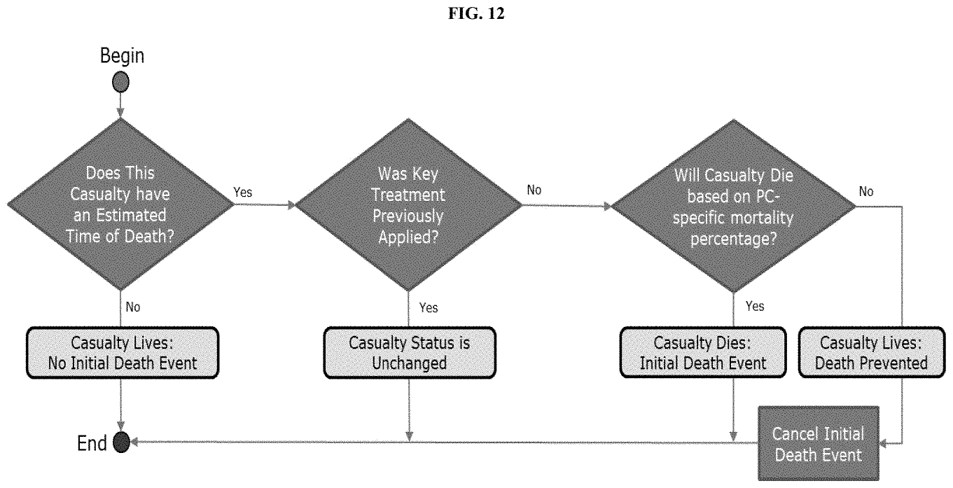

FIG. 11 shows the process for updating biological mortality based on treatment.

The flowchart starts when a biological casualty arrives at an MTF

### SUMMARY OF THE INVENTION

An objective of the present invention is a method to estimate patient conditions and effects for various chemical biological, radiological, and nuclear agents.

Another objective of the present invention is a method to develop estimates of medical requirements (i.e. MTF capabilities) needed, including but not limited to operating rooms (OR), intensive care unit (ICU), ward beds, Critical Care Air Transport Team (CCATT), and Class VIII consumable supplies needed, and evacuations and returns to duty estimates, based on anticipated patient loads.

Yet another objective of the present invention is a method for generating medical laydowns for a medical treatment network within a theater, including their spatial/geographic arrangements.

Yet another objective of the present invention is a method for modelling patient care in response to CBRN events, and for identifying the demands for medical care, casualty transportation requirements, and projecting patient clinical outcomes from the Point of Injury (POI) through evacuation from theater.

### DETAILED DESCRIPTION OF THE INVENTION

### Definition

Class VIII supply. The United States Army divides supplies into ten numerically identifiable classes of supply. Class VIII supplies are medical materials (equipment and consumables) including repair parts particular to medical equipment. Class VIIIa supplies are medical consumable supplies not including blood & blood products; Class VIIIb supplies are blood & blood components (whole blood, platelets, plasma, packed red cells, etc.).

Role 3 expeditionary medical requirements. In Role 3, the patient is treated in an Medical Treatment Facility (MTF) or veterinary facility (for working animals) that is staffed and equipped to provide care to all categories of patients, to include resuscitation, initial wound surgery, specialty surgery (general, orthopedic, urogenital, thoracic, ENT, neurosurgical) and post-operative.

Theater. A theater or theatre is an area in which important military events occur or are progressing. A theater can include the entirety of the airspace, land and sea area that is or that may potentially become involved in war operations. In this application, a theater can also include a civilian area where a CBRN event took place.

Role 1 medical facility provides primary healthcare, specialized first aid, triage, resuscitation, and stabilization. Normally included within the basic Role 1 capabilities are routine sick call and the management of minor sick and injured personnel who can immediately return to duty.

Role 2 medical facility provides a greater capability to resuscitate trauma patients than is available at Role 1. . . . Role 2 care has the capability to provide packed blood products, limited x-ray, laboratory, dental support, combat and operational stress control, PVTMED, and Role 2 veterinary medical and resuscitative surgical support.

Role 3 medical facility is normally provided at Division level and above. It includes additional capabilities, including specialist diagnostic resources, specialist surgical and medical capabilities, preventive medicine, food inspection, dentistry, and operational stress management teams when not provided at level 2.

The present invention describes the newly developed CBRN casualty modeling functionality within the Medical Planners Toolkit (MPTk) and the Joint Medical Planning Tool (JMPT). This new functionality will provide the DoD and civilian medical planning community for the first time with the capability to model CBRN casualties on the battlefield or in a civilian area, and their impacts on the medical system. Using CBRN casualty estimates either developed by the DoD Defense Threat Reduction Agency (DTRA) or existing as a pre-developed scenario within MPTk, medical logistic planners are able to develop a casualty stream (i.e. estimated incoming casualty flow into a medical network due to a CBRN event). The information of a casualty stream includes information such as casualty estimates, resulting patient conditions and effects for various chemical biological, radiological, and nuclear agents. These casualty streams can be used within MPTk to develop estimates of Role 3 expeditionary medical requirements for operating rooms (OR), intensive care unit (ICU) and ward beds, evacuations, returns to duty, Critical Care Air Transport Team (CCATT), and Class VIII consumable supplies based on anticipated patient loads.

The capability to develop estimates for casualty types and numbers, medical requirements, medical supplies and to simulate the treatment and movement of CBRN casualties in MPTk and JMPT will provide the DoD and civilian emergency medical personnel with the ability to
1) generate CBRN casualty estimates based on user input (typically provided by the DTRA Reachback team),
2) provide visibility into patient treatment and routing of CBRN casualties from POI to final disposition within the theater or evacuation out of the theater,
3) simulate health care provision for CBRN casualties from first responder through theater hospitalization,
4) model patient movement of CBRN casualties through a medical treatment network, and
5) provide a robust reporting capability.

This capability will also allow medical logistic planer to more effectively estimate and plan for the requirements necessary to support military operations in a conflict in which CBRN weapons may be employed, or to plan for a CBRN attack in a civilian area.

Both MPTk and JMPT have the ability to characterize the medical requirements necessary to treat casualties resulting from CBRN weapons use in sufficient detail to capture resource requirements within shore-based medical treatment facilities (MTF) and Casualty Receiving and Treatment Ships (CRTS). The following are the main components of the MPTk and JMPT CBRN capability (FIG. 1):

MPTk provides the capability to incorporate CBRN patient streams based on a variety of CBRN agents that may be employed by enemy combatants in ground combat and fixed base scenarios. The CBRN patient streams will typically be provided by DTRA Reachback and incorporated into the Casualty Rate Estimation Tool (CREstT), and/or the casualty estimation tool within MPTk. The imported CBRN casualty stream can be combined with the conventional casualty estimate to develop a more comprehensive estimate for hospitalization (theater hospital admissions, operating room requirements, intensive care unit (ICU), ward and staging beds, theater evacuation requirements, return to duty estimates, blood and supply requirements). These estimates allow medical planner to prepare and set up medical treatment facilities to meet these anticipated requirements.

Once the DTRA or MPTk CBRN casualty estimates are developed, they may be imported to the JMPT tool. The JMPT tool will then simulate patient flow through a user definable network of MTFs; accounting for arrival, waiting, treatment, travel times, and patient disposition as well as differentiating between killed in action (KIA) and died of wounds (DOW), and determine Return-to-Duty dispositions, all as a function of the user-define medical theater laydown. The JMPT model provides a robust reporting capability that allows the user to specially examine the CBRN casualty impacts on the medical network of the requirements to treat, transport, manage, and assess clinical outcomes of CBRN casualties. User can then adjust the set-up of the medical network to meet the demand of CBRN casualties and select the most suitable set-up plan, which offers the least estimated died of wounds (DOW), and best Return-to-Duty dispositions.

As described in previous applications, the MPTk combines the Patient Condition Occurrence Frequency (PCOF) tool, the Casualty Rate Estimation Tool (CREstT), the Expeditionary Medical Requirements Estimator (EMRE), and the Estimating Supplies Program (ESP) into a single desktop application (see FIG. 2). This allows the user to manage the frequency distributions of probabilities of illness and injury, estimate casualties in a wide variety of military scenarios, estimate medical requirements for theater hospitalization, and estimate supply usage.
Patient Condition Occurrence Frequency (PCOF) Tool is capable of generation and management of patient condition occurrence distributions;
Casualty Rate Estimation Tool (CREstT) provides estimation of casualties in ground-combat, fixed-based, and shipboard environments;
Expeditionary Medical Requirements Estimator (EMRE) provides estimation of Role 3 hospitalization, evacuation, and evacuation requirements based on anticipated patient loads; and
Estimating Supplies Program (ESP) generates estimation of supply usage on a daily basis, including estimates of weight and volume of the supplies.

JMPT is a software program designed for medical planners as a simulation tool that models the flow of patients from the point of injury through more definitive care. JMPT is also an operations research tool that supports systems analysis, operational risk assessment, and field medical services planning. It incorporates extensive data for over 300 patient conditions and their corresponding medical treatment tasks, treatment times, critical equipment, transportation assets, medical personnel and required skills, and levels of care. It employs a Monte Carlo method to simulate patient losses due to treatment delays and medical complications, to assess the impact that resource limitations, system bottlenecks, skill limitations, and other factors have on them.

JMPT can:
Generate patient conditions for a patient stream in an identified theater.
Prioritize treatment and evacuation routing of patients based on severity of injuries.
Model mortality as killed in action (KIA) and died of wounds (DOW) as a function of time.
Simulate patient flow through a network of medical facilities, including arrival times, wait times, and treatment times.
Offer the ability to build a generic treatment facility with user-defined medical capability and personnel assets.
Model routing and utilization of transportation assets.
Provide dynamic reports in graph and tabular formats that show medical treatment facility (MTF) status, patient disposition, and resource utilization.

In the present invention, CRETst module of MPTk is changed to include three categories of patients: conventional injuries only; CBRN-related injuries only; and the combination of a conventional injury with a CBRN-related exposure. While CREstT is used to estimate the number of conventional casualties, the number of CBRN casualties will not be estimated in CREstT. They will be estimated using the JEM, a DoD validated tool used to predict and track Nuclear, Biological, and Chemical events and effects.

Incorporate casualty estimates from JEM into a CREstT scenario. JEM is capable of providing all warfighters with the ability to accurately model and predict the time-phased impact of CBRN and Toxic Industrial Chemical/Material events and effects. It incorporates the impacts of weather, terrain, and material interactions into the downwind prediction.

The data imported into MPTk must contain the number of casualties, the agent involved in the CBRN attack, and injury severity. The type of attack and severity level are also required for each casualty because these data are used in CREstT to determine the correct PC code and treatment profiles to associate with the casualty.

TABLE 1
An example of JEM casualty table.

| | | | | | | | | Very | |
|---|---|---|---|---|---|---|---|---|---|
| Group | Day | DTG | Location | Agent | Mild | Moderate | Severe | Severe | KIA |
| Close area | 2 | Aug. 9, | 42.1583° N, | GD | 73 | 59 | 3 | 2 | 12 |
| | | 2016 13:23 | 41.6714° E | | | | | | |
| Support area (FWD) | 5 | Aug. 14, | 51.2456° N, | VX | 79 | 47 | 35 | 17 | 2 |
| | | 2016 2:34 | 49.3424° E | | | | | | |
| Support area (REAR) | 11 | Aug. 25, | 54.9382° N, | Sarin | 73 | 55 | 28 | 9 | 4 |
| | | 2016 15:56 | 68.5458° E | | | | | | |
| Close area | 12 | Sep. 6, | 56.9524° N, | Sarin | 29 | 54 | 38 | 8 | 2 |
| | | 2016 11:59 | 24.1241° E | | | | | | |
| Close area | 17 | Sep. 23, | 34.5553 N, | VX | 84 | 52 | 23 | 11 | 1 |
| | | 2016 20:34 | 69.2075 E | | | | | | |

An example of injury severity table from JEM is depicted in Table 2, which is amended to match the categories used in the CBRN PC Code mapping table.

TABLE 2
An example of injury severity table.

| | Degree | Description |
|---|---|---|
| 1 | Mild | Injury is manifesting symptoms (and signs for biological agents) of such severity that |
| | | individuals can care for themselves or be helped by untrained personnel. Condition may not |
| | | impact the ability to conduct the assigned mission. |
| 2 | Moderate | Injury is manifesting symptoms (and signs for biological agents) of such severity that |
| | | medical care may be required. General condition permits treatment as out patient and some |
| | | continuing care and relief of pain may be required before definitive care is given. Condition |
| | | may be expected to interrupt or preclude the ability to conduct the assigned mission. |
| 3 | Severe | Injury is manifesting symptoms (and signs for biological agents) of such severity that there |
| | | is cause for immediate concern, but there is no immediate danger to life. Individual is acutely |
| | | ill and likely reguires hospital care. Indicators are questionable-condition may or may not |
| | | reverse without medical intervention. Individual is unable to conduct the assigned mission |
| | | due to severity of injury. |
| 4 | Very severe | Injury is manifesting symptoms (and signs for biological agents) of such severity that life is |
| | | immediately endangered. Indicators are unfavorable-condition may or may not reverse, |
| | | even with medical intervention. Prognosis is death without medical intervention. Individual is |
| | | unable to conduct the assigned mission and is not expected to retore to the mission due to |
| | | severity of injury. |

CREstT Changes. In the present invention, a mapping table was developed within CREstT that assigns a PC code to each CBRN casualty imported from JEM, based on the agent type and injury severity level. A new CBRN PC code shall be developed for each combination of CBRN agent and severity level and included in the PC code mapping table. Additionally (where applicable), the incubation times for each CBRN agent and severity level will be included in the table. Data is needed that describes the average time and distribution for these incubation periods.

In an embodiment of the present invention, a medical planer can include CBRN casualties in a CREstT scenario. CREstT allow the user to select the method for calculating the number of patients with multiple injuries (i.e. one conventional injury and one CBRN injury). The user can choose to allow CREstT to calculate the percentage of CBRN casualties with conventional injuries during runtime or can choose to specify the percentage manually. If the manual method for combining injuries is selected, the user can chose the percentage of CBRN casualties that will also have a conventional injury.

If the calculate function is selected in CREstT, CREstT will determine a percentage for each group and day during runtime by assuming the likelihood of receiving a conventional injury and the likelihood of receiving a CBRN injury are independent. Therefore, CBRN casualties should receive conventional injuries in the same proportions as the overall population. The percentage of the PAR that receives an injury of each patient type [i.e. wounded in action (WIA), disease (DIS), trauma (TRA), or nonbattle injury (NBI)] will be used as the percentages of CBRN patients that receive multiple injuries. For each day that a CBRN attack occurs, CREstT will generate conventional casualties as usual, calculate the PAR percentage that had injuries of each patient type on that day, calculate the number of CBRN patients that have multiple injuries of each patient type, which is equal to the PAR percentage that has each patient type multiplied by the total number of CBRN casualties. For example, assume a PAR of 10,000 on the day of a CBRN attack. CREstT ground combat produces 20 WIA, 10 NBI, 30 DIS, and the CBRN attack produces 1000 CBRN casualties. WIA casualties are 0.2% of PAR (20/10000), NBI are 0.1% of PAR (10/10000) and DIS are 0.3% of PAR (30/10000). These percentages will be applied to the CBRN casualties, producing two CBRN multi-injuries with a conventional WIA injury (0.2%*1000), one NBI multi injury (0.1%*1000), and three DIS multi-injury (0.3%*1000).

The Ground Combat Scenario allows the user to specify the PAR for various phases of the battle. CREstT determines the number of WIA, DIS, NBI, and/or TRA that occurred during that phase of the operation. The same PAR is input into JEM if a CBRN event is modeled to determine how many CBRN casualties occurred. Since the same PAR is exposed to combat and the CBRN event, a percentage of the PAR will suffer both types of injury.

The CBRN casualties stream will then be generated and added to the conventional patient stream. FIG. 3 represents an example of the process flow for integrating the CBRN casualty estimate from JEM with the conventional patient stream in CREstT. The procedures are described in the following bullets:
Add a new line to the Patient Stream for each type CBRN injury (Sarin Mild, Sarin Moderate, etc.) and indicate the number of WIA casualties aligned with that PC code on the appropriate day in the appropriate Casualty Group.
If the incubation period is modeled, the incubation time is added to the JEM day and start time and could possibly move the CBRN injury into a later day.
In the patient stream, the user specified or calculated number of multiple injuries will be modeled by randomly appending a CBRN injury to the right type of conventional injury for the day of the CBRN attack. The conventional injury doesn't necessarily have to be WIA (could be NBI with a CBRN injury, for example).
Determine how many multiple injuries are needed (user specified or calculated percentage of chemical injuries)
Randomly select a conventional ICD-9 code that has a casualty for that day, and then randomly select a CBRN PC code that also has a casualty for that day.
Create a new line in the patient stream by appending the CBRN PC code to the conventional ICD-9 code. An example of CBRN PC Code Mapping Table is shown as Table 3.
Make the count 1 for this line, while decrementing the count in the individual conventional and CBRN lines.
Repeat this process until the appropriate number of multiple injuries is created. If the line with appended PC codes already exists, a new line is not created; the count is just incremented to 2, 3, . . .

TABLE 3
CBRN PC Code Mapping Table

```
Major Category
  Chemical
    Subcategory
```

| | −Sarin | Severity | PC | Description | Incubation period (min) |
|---|---|---|---|---|---|
| | | Mild | | Mild Sarin Contaminant | 60 |
| | | Moderate | | Moderate Sarin Contaminant | 45 |
| | | Severe | | Severe Sarin Contaminant | 30 |
| | | Very severe | | Very Severe Sarin Contaminant | 15 |
| | −GD | Severity | PC | Description | Incubation period (min) |
| | | Mild | | Mild GD Contaminant | 60 |
| | | Moderate | | Moderate GD Contaminant | 45 |
| | | Severe | | Severe GD Contaminant | 30 |
| | | Very severe | | Very Severe GD Contaminant | 15 |
| Biological | +Anthrax | Severity | PC | Description | |
| Radiological | +Uranium 225 | Severity | PC | Description | |
| Nuclear | +Uranium 225 | Severity | PC | Description | |

CREstT allows the user to export a patient stream using the JMPT Replication Patient Stream Generator. The JMPT Table Patient Stream Generator and the Statistic-based Single Replication Patient Stream Generator will not be active if CBRN PC codes exist in the patient stream.

CREstT shall use the JEM Input File and the information gathered from the user input to assign times to each CBRN or multi-injury casualty that falls within the attack window. For example (as shown in FIG. 4) assuming that modeling the incubation period is not selected, there are 3 very severe GD casualties that occurred on Day 2 starting at 13:23. The attack lasted for 32 minutes. CREstT shall select 3 of the GD casualties from the patient stream and assign times spaced between the start and end time of the GD attack. In this case, one of the GD casualties selected also has a conventional injury. This technique is repeated until every CBRN or multi-injury casualty in the patient stream is assigned times based on the user specified window. Then all the conventional injuries are spaced apart uniformly distributed over the entire day. If modeling the incubation period was selected, the incubation time would be added to the GD CBRN or multi-injury casualties to ensure they wouldn't enter the medical facility until symptoms became obvious.

ESP CBRN Modifications. ESP estimates the quantities of consumable medical supplies necessary to treat a patient stream. ESP integrates with CREstT, uses time-phased patient streams produced by CREstT scenarios to generate estimates of supply usage on a daily basis and includes estimates of cost, weight, and volume. For each supply necessary for treating CBRN casualties, that is not established in MTPk database, information is developed, which include 1) number of individual units of measure inside one unit of issue for a supply, 2) cost of one unit of issue (UI) of a supply, 3) weight of one UI of supply, 4) volume of one UI of supply.

In one embodiment of the present invention, ESP simulate the treatment of patients with CBRN injuries based on the new CBRN PC codes. While the methodology for simulating the treatment of patients with CBRN injuries may be the same as the existing methodology, the data will be different. Any special equipment needed to treat CBRN injuries must be identified and paired with the appropriate PC codes. For each new CBRN PC code, a treatment brief and treatment profiles shall be developed, which consisting of:1) Probability that patient returns to duty after treatment at Role 1; 2)Probability that patient returns to duty after treatment at Role 2; 3) Probability of surgery; 4) Average Length of Stay (LOS) parameters, 5) Percent chance patient will visit Functional Area (FA); 6) Average Length of Stay in FAPercent chance task will be performed in FA, 7) Percent chance task will be repeated in FA, 8) Number of times task is typically repeated at FA in first 24 hours, 9) Percent chance patient will use supplies in supply collection for task in FA, 10) Number of supply blocks patient will use for task in FA, and 11)Quantity of supply used in the supply block.

In another embodiment of the present invention, ESP generates estimates for medical supplies to accommodate the combined treatment profiles resulting from multiple-injury casualties. The methodology considers the combined requirements for treating both injuries. For example, ESP will encounter patients that may require separate surgeries for both injuries. The patient cannot Return to Duty until the recovery requirements for both surgeries have been met. CREstT will have the ability to combine treatment profiles for conventional and CBRN injuries, in response to the portion of the patients in the Patient Stream having multiple injuries. ESP will necessarily have the capability to process multiple-injury patients.

EMRE modification. The Expeditionary Medical Requirements Estimator (EMRE) of MPTk estimates the operating room, ICU bed, ward bed, evacuation, and blood product requirements for theater hospitalization based on a given patient load. EMRE can provide these estimates based on a user-specified average daily patient count, or it can use the patient streams derived by CREstT as EMRE is fully integrated with both CREstT and the PCOF tool. EMRE also uses stochastic processes to allow users to evaluate risk in medical planning.

In an embodiment of the present invention, the EMRE module is modified to simulate the treatment of patients with CBRN injuries (who will have one of the new CBRN PC codes). While the methodology for simulating the treatment of patients with CBRN injuries may be the same as the existing methodology, the data will be different. Any special requirements for treating CBRN injuries must be identified and paired with the appropriate PC codes. For each new CBRN PC code, the following data shall be developed, which include: 1) Probability that a patient becomes a theater hospitalization; 2) Probability of surgery, 3) Recurrence interval (time in days between the 1st surgery and recurring surgeries), 4) Incubation period (time it takes for symptoms to manifest after exposure), 5) Average initial surgery duration in minutes 6) Average follow-up surgery duration in minutes, 7) Length of Stay (LOS) parameters: a) amount of time in days a patient spends in: ICU (After surgery, Without surgery) and Ward (After surgery, Without surgery). New planning factors are added for Medical Counter Measure medications

In another embodiment of the present invention, EMRE accommodates the combined treatment profiles resulting from multiple-injury casualties. A new methodology considers the combined requirements for treating both injuries. For example, EMRE will encounter patients that may require separate surgeries for both injuries. Surgery and recovery times for both injuries must be considered in combination. CREstT will have the ability to combine conventional and CBRN injuries, resulting in a portion of the patients in the Patient Stream having multiple injuries. EMRE will necessarily have the capability to process multiple-injury patients.

JMPT modification. JMPT is a software program designed for medical planners to model the flow of patients from the point of injury through more definitive care. JMPT is also an operations research tool that supports systems analysis, operational risk assessment, and field medical services planning. JMPT incorporates extensive data for over 300 patient conditions and their corresponding medical treatment tasks, treatment times, critical equipment, transportation assets, medical personnel and required skills, and levels of care. It employs a Monte Carlo method to simulate patient losses due to treatment delays and medical complications, to assess the impact that resource limitations, system bottlenecks, skill limitations, and other factors have on them.

The PC codes and associated data for CBRN agents are added in the JMPT Common Database. A different PC Code is assigned to each agent and severity level combination. A category and a subcategory are assigned to each CBRN entry in the JMPT Common Database. The categories are chemical, biological, radiological, or nuclear and the subcategories are the name of the agent. See Table 4. Treatment profiles shall be developed for each of these CBRN PCs.

TABLE 4
JMPT PC Codes

| | | | | | |
|---|---|---|---|---|---|
| Patient Codes | Task Types | Equipment Types | Personnel Types | Role 1 DOW Coefficients | Role 2-3 DOW Coefficients |

| Patient Code [symbol] | Category [symbol] | Subcategory [symbol] | Description [symbol] |
|---|---|---|---|
| 850.1 | Chemical | Sarin | Sarin Mild Symptoms |
| 850.2 | Chemical | Sarin | Sarin Moderate Symptoms |
| 850.3 | Chemical | Sarin | Sarin Severe Symptoms |
| 850.4 | Chemical | Sarin | Sarin Very Severe Symptoms |
| 851.1 | Biological | Anthrax | Anthrax Mild Symptoms |
| 851.1 | Biological | Anthrax | Anthrax Moderate Symptoms |
| 851.1 | Biological | Anthrax | Anthrax Severe Symptoms |
| 851.1 | Biological | Anthrax | Anthrax Very Severe Symptoms |
| 852.1 | Radiological | RDD | RDD Mild Symptoms |
| 852.1 | Radiological | RDD | RDD Moderate Symptoms |
| 852.1 | Radiological | RDD | RDD Severe Symptoms |
| 852.1 | Radiological | RDD | RDD Very Severe Symptoms |
| 853.1 | Nuclear | U235 | U235 Mild Symptoms |
| 853.1 | Nuclear | U235 | U235 Moderate Symptoms |
| 853.1 | Nuclear | U235 | U235 Severe Symptoms |
| 853.1 | Nuclear | U235 | U235 Very Severe Symptoms |

JMPT combines treatment profiles for two injuries during runtime. Patient streams from scenarios with CBRN casualties will have patients with both CBRN-related and conventional injuries. JMPT will compile ad hoc treatment profiles for a combination of injuries. The treatment profiles in the common database are based on a single injury case. An algorithm maybe developed to combine these into multi-injury treatment profiles, as needed. As an example using two conventional injuries, Table 5 shows three different treatment profiles. The first column shows the treatment profile for Injury #1 (open wound of the head, face or neck, deep, extensive, into muscle). The second column shows the treatment profile for Injury #2 (fracture at or below elbow (Radius, Ulna, Carpal, Metacarpal or Phalanges), unilateral). The third column shows the treatment profile for a casualty with both injuries.

TABLE 5
Multi-Injury Treatment Profiles

| Injury 1 | Injury 2 | Combined Treatment Profile |
|---|---|---|
| REMOVE AND COLLECT BELONGINGS, | REMOVE AND COLLECT BELONGINGS, | REMOVE AND COLLECT BELONGINGS, |
| WEAPONS AND EQUIPMENT | WEAPONS AND EQUIPMENT | WEAPONS AND EQUIPMENT |
| TRIAGE | TRIAGE | TRIAGE |
| ASSESSMENT AND EVALUATION | ASSESSMENT AND EVALUATION | ASSESSMENT AND EVALUATION |
| OF PATIENT STATUS | OF PATIENT STATUS | OF PATIENT STATUS |
| VITAL SIGNS | VITAL SIGNS | VITAL SIGNS |
| NEUROLOGICAL ASSESSMENT | | NEUROLOGICAL ASSESSMENT |
| STABILIZE SPINE | | STABILIZE SPINE |
| (COLLAR/SPINE BOARD) | | (COLLAR/SPINE BOARD) |
| ESTABLISH ADEQUATE AIRWAY | | ESTABLISH ADEQUATE AIRWAY |
| (ORO/NASO PHARYNGEAL ONLY) | | (ORO/NASO PHARYNGEAL ONLY) |
| EMERGENCY CRICOTHYROIDOTOMY | | EMERGENCY CRICOTHYROIDOTOMY |
| (5% chance) | | (5% chance) |
| O2 ADMINISTRATION CONTINUOUS | | O2 ADMINISTRATION CONTINUOUS |
| (NASAL/MASK) | | (NASAL/MASK) |
| CARDIO ARREST RESUSCITATION | | CARDIO ARREST RESUSCITATION |
| (50% chance) | | (50% chance) |
| PERFORM VENTILATION WITH | | PERFORM VENTILATION WITH |
| BAG VALVE MASK | | BAG VALVE MASK |
| CONTROL BLEEDING | | CONTROL BLEEDING |
| CIRCULATION CHECK | | CIRCULATION CHECK |
| | CIRCULATION CHECK | CIRCULATION CHECK |
| START/CHANGE IV INFUSION SITE | START/CHANGE IV INFUSION SITE | START/CHANGE IV INFUSION SITE |
| ADMINISTER IV FLUID | ADMINISTER IV FLUID | ADMINISTER IV FLUID |
| OBTAIN SPECIMEN FOR | | OBTAIN SPECIMEN FOR |
| LABORATORY ANALYSIS | | LABORATORY ANALYSIS |
| ACTIVE PATIENT REWARMING | | ACTIVE PATIENT REWARMING |
| | DOPPLER ASSESSMENT | DOPPLER ASSESSMENT |
| | (25% chance) | (25% chance) |
| CATHETERIZATION FOLEY | CATHETERIZATION FOLEY | CATHETERIZATION FOLEY |
| (10% chance) | (10% chance) | (10% chance) |
| MEASURE/RECORD INTAKE/OUTPUT | MEASURE/RECORD INTAKE/OUTPUT | MEASURE/RECORD INTAKE/OUTPUT |
| INTERPRET LAB RESULTS | | INTERPRET LAB RESULTS |
| ORDER AND DOCUMENT | ORDER AND DOCUMENT | ORDER AND DOCUMENT |
| APPROPRIATE MEDS/TREATMENT | APPROPRIATE MEDS/TREATMENT | APPROPRIATE MEDS/TREATMENT |

Note that some of the medical tasks are combined and some are repeated. For example, the first four tasks (remove belongings, triage, assessment, and vital signs) each only appear once in combined profile, despite the fact that they appear in both of the other profiles. This is because these tasks only need to be performed once no matter how many injuries the casualty has. However, further down in the profile the tasks for circulation check (highlighted in orange) are repeated in the combined profile because a different circulation check is needed for each of the injuries. Note also that the tasks are interleaved in an appropriate sequence. Each task has a sequence ID (not shown) to make sure that the tasks are performed in the correct order. Injury-based data such as Minimum Time to RTD, Stabilization Time, Mortality Risk, or Ambulatory/Litter will need to be consolidated for multiple injuries.

JMPT also includes User-Defined and Replication-based Patient Stream Generators, which support the combination of two injuries per casualty and number of a particular casualty type created at a certain time. An additional field, “#Casualties”, allows the user to specify how many of the same type injuries were generated at that time. This eliminates the need to create the same type injury multiple times for a CBRN or mass casualty event.

Mortality modeling of JMPT uses role of care and patient code-specific mortality percentages from the Joint Readiness Clinical Advisory Board to determine which casualties die at each role of care. To stimulate CBRN casualty event, JMPT integrates two different mortality curves. A standard mortality curves for conventional injuries and a new set of mortality curves may be developed for CBRN casualties based on mortality data contained in the Allied Medical Publication (AMedP-8). JMPT applies the correct curve based on PC code. Further, JMPT may adapt the current DOW methodology to combine the effects of conventional mortality risk with that associated with CBRN injuries to emulate the combined effects of both injuries. The time to death will be different for a casualty with conventional injury than one suffering from a CBRN injury only and will be different still for a casualty with both types of injury. Using two different curves will provide more accurate DOW results in the case of single injuries of either type while extension of the current DOW methodology will allow for emulation of the combined effects when injuries of both types are present in a single patient.

The chemical mortality model can be broken down into two segments: (1) Role 1 and (2) higher roles of care. For Role 1 chemical mortality, JMPT will use the initial (i.e., no treatment) time of death and the Role 1 death percentages for each patient code. Upon generation of each casualty, JMPT will use the role-specific death percentages to determine randomly if that casualty should die at Role 1. If the first facility the casualty enters is a higher role of care, the Role 1 mortality decision will be ignored and the casualty will proceed with the higher role of care mortality logic. If the casualty is determined to die at Role 1, a death event will be scheduled and the casualty will be removed from the system at the initial time of death, assuming that time occurs before the casualty attempts evacuation from Role 1 to a higher role of care. If the casualty attempts evacuation to a higher role of care prior to the initial time of death occurring, the casualty will die and be removed from the system upon requesting evacuation. If the casualty is determined not to die at Role 1, the casualty will proceed until either returning to duty or evacuating to a higher role of care. Role 1 mortality algorithms are shown in FIG. 5

Decontamination facilities were also created for JMPT. Users will place decontamination facilities before each military treatment facility (MTF) where CBRN casualties might be received. Decontamination of CBRN casualties will be simulated at these MTFs. The amount of time required for the task will vary, and depends on the level of care, level of decontamination, and type of agent. See table 6.

TABLE 6
Decontamination of Personnel

| Levels | Techniques | Responsible |
|---|---|---|
| Immediate | Skin decontamination | Individual |
| | Personal wipe down | Individual/buddy |
| | Operator wipe down | Individual/crew |
| | Spot decantaminatoin | Individual/crew |
| Operational | MOPP gear exchange | Unit |
| | Vehicle wash down | Battalion crew or |
| | | decontamination platoon |
| Thorough | DTD | Contaminated unit with |
| | | assistance from CBRN unit |
| | DED/DAD | Decontamination platoon |
| Clearance | Unrestricted use of | Supporting strategic resources |
| | resources | |

Note.
DAD = detailed aircraft decontamination, DED = detailed equipment decontamination, DTD = detailed troop decontamination.

Data do not currently exist to replicate the time-based mortality model utilized for conventional casualties, so a different method is adopted to generate CBRN casualties. The Defense Threat Reduction Agency (DTRA) maintains role of care and patient code specific death percentages for use in CBRN casualty estimating tools. These are represented as the percentage of each patient code at each role of care that will die at that role of care. JMPT uses these estimates to approximate mortality. However, different types of CBRN injuries use different mechanisms to model key treatment events and mortality risk progression. Each of these cases are described below.

### JMPT CBRN Reports

JMPT shall create new reports or modify existing reports to view CBRN results. Medical planners must be able to view output and charts based on the categories and subcategories so that he can extract data on metrics such as number of CBRN patients treated, transported, DOW, RTD, etc.

### Mortality Modeling in JMPT

Data do not currently exist to replicate the time-based mortality model utilized for conventional casualties, so a different method is adopted for CBRN casualties in the present invention. The Defense Threat Reduction Agency (DTRA) maintains role of care and patient code specific death percentages for use in CBRN casualty estimating tools. These are represented as the percentage of each patient code at each role of care that will die at that role of care. JMPT uses these estimates to approximate mortality. However, different types of CBRN injuries use different mechanisms to model key treatment events and mortality risk progression.

### Chemical Casualties Mortality Model

The chemical mortality model can be broken down into two segments: (1) Role 1 medical facilities and (2) higher roles of care. For Role 1 chemical mortality (FIG. 8), JMPT will use the initial (i.e., no treatment) time of death and the Role 1 death percentages for each patient code. Upon generation of each casualty, JMPT will use the role-specific death percentages to determine randomly if that casualty should die at Role 1. If the first facility the casualty enters is a higher role of care, the Role 1 mortality decision will be ignored, and the casualty will proceed with the higher role of care mortality logic (FIG. 9). If the casualty is determined to die at Role 1, a death event will be scheduled, and the casualty will be removed from the system at the initial time of death, assuming that time occurs before the casualty attempts evacuation from Role 1 to a higher role of care. If the casualty attempts evacuation to a higher role of care prior to the initial time of death occurring, the casualty will die and be removed from the system upon requesting evacuation. If the casualty is determined not to die at Role 1, the casualty will proceed until either returning to duty or evacuating to a higher role of care. Role 1 mortality algorithms are shown in FIG. 8.

For higher roles of care, JMPT will use only the role of care and patient code specific death percentages to approximate mortality. Upon attempted evacuation to a higher role of care, JMPT will use the role-specific death percentages to determine randomly if the casualty should die. If the casualty is supposed to die, the casualty will be removed from the system prior to evacuating. If the casualty isn't supposed to die, the casualty will evacuate to the next role of care. Only casualties scheduled to evacuate at each MTF will be subject to the mortality logic. To ensure coverage of the highest role of care the casualty visits in the scenario, the mortality logic will always be evaluated at final evacuation. Collection points are considered to be equal to the previously visited role of care so casualties evacuating to a collection point will never evaluate the mortality logic. Higher role of care mortality algorithms are shown in FIG. 9.

### Modeling Biological Casualties Mortality

Casualties with biological injuries can degrade to a more serious patient condition if key treatment is not received quickly enough. FIG. 10 shows an example of a PSG with biological casualties. “PC” is the casualty's initial condition and “PC 2” is the condition to which the casualty could potentially degrade. “PC2 Injury Time” is the simulation time after which the casualty could degrade.

Degradation can be prevented if a casualty receives treatment at an MTF with the correct key treatment supply item for the casualty's condition. Currently, the key supply items supported in JMPT are antitoxins and antibiotics. Key Treatment is applied when the casualty arrives at an MTF, when the casualty has completed treatment at an MTF and is ready to evacuate, or when the casualty arrives at an incoming Staging Facility. Once key treatment is applied to a casualty, that casualty can no longer degrade to a serious patient condition, but they can still die. Each biological patient condition has a “treated probability of death” in the JMPT common data. Once key treatment has been applied, a random draw is compared to the treated probability of death to determine if the casualty's death has been prevented.

If treatment does not prevent a biological casualty's death, it can still change the estimated time of death. The following biological patient condition codes will randomly draw from a lognormal distribution to update the casualty's estimated time of death after treatment:
[symbol] THRAX EARLY
ANTHRAX LATE
PLAGUE LATE

The remaining biological patient codes (e.g., BOT EARLY, BOT LATE, etc.) do not update the estimated time of death if treatment is ineffective.

FIG. 11 shows the process for updating biological mortality based on treatment. The flowchart starts when a biological casualty arrives at an MTF. There is one additional piece of logic not shown in FIG. 11 that applies to the following patient codes:
ANTHRAX LATE
BOT LATE
PLAGUE LATE
VHF

If a casualty with one of these patient codes has a treated death probability and no key treatment supply specified, then key treatment is assumed to occur when the casualty is treated at an MTF with Role 3 or higher care. This treatment affects mortality in the same way as receiving key treatment supplies as described above.

### Modeling Radiological and Nuclear Injuries Mortality

Some radiological and nuclear patient codes use the conventional mortality model because the conventional portion of the injury is the dominant factor for mortality. For these casualties, the mortality model for the most relevant NHRC conventional patient code (either trauma or burn) was used as a baseline for mortality risk (see table 8). The total number of non-surviving casualties under a variety of potential scenario timelines were assessed. These scenario boundary conditions represented the reasonable range of patient flow timelines that could be seen in theater level operational planning scenarios. The mortality risk breakdown for the baseline injury was then adjusted with the goal of targeting, on average amongst these scenarios, a 2× increase in non-surviving casualties for moderate radiation combination injuries and a 3× increase in mortality for severe radiation combination injuries. The mild radiation combination injuries use the conventional model without an adjustment. Since the total number of deaths depends on the treatment time at each role of care, these 2× and 3× adjustments can vary, depending on the scenario. These factor adjustments were recommended by the Armed Forces Radiobiology Research Institute (AFRRI) Military Medical Operations Division. The radiological/nuclear patient codes that use this model to estimate mortality can be found in 0.

TABLE 7
Radiological/Nuclear Mortality - Conventional Models

| | | Closest | |
|---|---|---|---|
| | | Baseline | |
| | | Conventional | Adjustment |
| Code | Description | Patient Code | Factor |
| RC04 | Lacerations: (open wounds | 879.6 | Unadjusted |
| | without fractures) with Radiation | | |
| | Injury (0.7-1.25 Gy) | | |
| RC05 | Lacerations: (open wounds | 879.6 | 2x |
| | without fractures) with Radiation | | |
| | Injury (>1.25-3.0 Gy) | | |
| RC07 | Thoracic/abdominal tissue | 860.5 | Unadjusted |
| | trauma: (wound thorax, open, | | |
| | lacerate, contused) with | | |
| | Radiation Injury (0.7-1.25 Gy) | | |
| RC08 | Thoracic/abdominal tissue | 860.5 | 2x |
| | trauma: (wound thorax, open, | | |
| | lacerate, contused) with | | |
| | Radiation Injury (>1.25-3.0 Gy) | | |
| RC09 | Thoracic/abdominal tissue | 860.5 | 3x |
| | trauma: (wound thorax, open, | | |
| | lacerate, contused) with | | |
| | Radiation Injury (>3.0-6.0 Gy) | | |
| RC13 | Extremity trauma open: (fracture | 812.31 | Unadjusted |
| | humerus) with Radiation Injury | | |
| | (0.7-1.25Gy) | | |
| RC14 | Extremity trauma open: (fracture | 812.31 | 2x |
| | humerus) with Radiation Injury | | |
| | (>1.25-3.0 Gy) | | |
| RC16 | Extremity trauma closed: | 821.00 | Unadjusted |
| | (fracture closed femur) with | | |
| | Radiation Injury (0.7-1.25 Gy) | | |
| RC17 | Extremity trauma closed: | 821.00 | 2x |
| | (fracture closed femur) with | | |
| | Radiation Injury (>1.25-3.0 Gy) | | |
| RC18 | Extremity trauma closed: | 821.00 | 2x |
| | (fracture closed femur) with | | |
| | Radiation Injury (>3.0-6.0 Gy) | | |
| RC22 | Abdominal fracture: (wound | 808_863 | Unadjusted |
| | abdomen open with pelvic | | |
| | fracture) with Radiation | | |
| | Injury (0.7-1.25 Gy) | | |
| RC23 | Abdominal fracture: (wound | 808_863 | 2x |
| | abdomen open with pelvic | | |
| | fracture) with Radiation | | |
| | Injury (>1.25-3.0 Gy) | | |
| RC24 | Abdominal fracture: (wound | 808_863 | 3x |
| | abdomen open with pelvic | | |
| | fracture) with Radiation | | |
| | Injury (>3.0-6 0 Gy) | | |
| RC25 | Mild Burns (2nd degree 6- | 942.20 | Unadjusted |
| | 16% TBSA) with Radiation | | |
| | Injury (0.7-1.25 Gy) | | |
| RC26 | Mild Burns (2nd degree 6- | 942.20 | 2x |
| | 16% TBSA) with Radiation | | |
| | Injury (>1.25-3.0 Gy) | | |
| RC28 | Moderate Burns (2nd degree >16- | 942.00 | Unadjusted |
| | 20% TBSA) with Radiation | | |
| | Injury (0.7-1.25 Gy) | | |
| RC29 | Moderate Burns (2nd degree >16- | 942.00 | 2x |
| | 20% TBSA) with Radiation | | |
| | Injury (>1.25-3.0 Gy) | | |
| RC30 | Moderate Burns (2nd degree >16- | 942.00 | 3x |
| | 20% TBSA) with Radiation | | |
| | Injury (>3.0-6.0 Gy) | | |
| RC31 | Severe Burns (2nd degree >20- | 942.30 | Unadjusted |
| | 44% TBSA*) with Radiation | | |
| | Injury (0.7-1.25 Gy) | | |
| RC32 | Severe Burns (2nd degree >20- | 942.30 | 2x |
| | 44% TBSA*) with Radiation | | |
| | Injury (>1.25-3.0 Gy) | | |
| RC33 | Severe Burns (2nd degree >20- | 942.30 | 3x |
| | 44% TBSA*) with Radiation | | |
| | Injury (>3.0-6.0 Gy) | | |
| RC34 | Moderate Burns (2nd degree >16- | 942.00 | Unadjusted |
| | 20% TBSA)/Lacerations: (open | | |
| | wounds without fractures) | | |
| | with Mild Radiation Injury | | |
| | (0.7-1.25 Gy) | | |
| RC35 | Lacerations/Mild Burns (2nd | 942.20 | 2x |
| | degree 6-16% TBSA) with | | |
| | Moderate Radiation Injury | | |
| | (1.25-3.0 Gy) | | |
| RC36 | Moderate Burns (2nd degree >16- | 942.00 | 2x |
| | 20% TBSA)/Lacerations: (open | | |
| | wounds without fractures) | | |
| | with Moderate Radiation Injury | | |
| | (>1.25-3.0 Gy) | | |
| RC37 | Head trauma/Mild Burns | 942.00 | Unadjusted |
| | (2nd degree 6-16% TBSA) | | |
| | with Radiation Injury | | |
| | (0.7-1.25 Gy) | | |
| RC38 | Head trauma/Moderate Burns | 942.00 | Unadjusted |
| | (2nd degree >16-20% TBSA) | | |
| | with Radiation Injury | | |
| | (0.7-1.25 Gy) | | |
| RC39 | Head trauma/Moderate Burns | 942.00 | 2x |
| | (2nd degree >16-20% TBSA) | | |
| | with Radiation Injury | | |
| | (>1.25-3.0 Gy) | | |
| RC40 | Extremity fracture/Mild | 942.20 | 2x |
| | Burns (2nd degree 6- | | |
| | 16% TBSA) with Moderate | | |
| | Radiation Injury | | |
| | (>1.25-3.0 Gy) | | |
| RC41 | Thoracic/abdominal tissue | 860.5 | Unadjusted |
| | trauma/Mild Burns (2nd | | |
| | degree 6-16% TBSA) with | | |
| | Mild Radiation Injury | | |
| | (0.7-1.25 Gy) | | |
| RC42 | Thoracic/abdominal tissue | 879.6 | Unadjusted |
| | trauma/Moderate Burns (2nd | | |
| | degree >16-20% TBSA) with | | |
| | Mild Radiation Injury | | |
| | (0.7-1.25 Gy) | | |
| RC43 | Lacerations: (open wounds | 942.20 | Unadjusted |
| | without fractures)/Mild | | |
| | Burns (2nd degree 6- | | |
| | 16% TBSA) | | |
| RC44 | Lacerations: (open wounds | 942.00 | Unadjusted |
| | without fractures)/Moderate | | |
| | Burns (2nd degree >16- | | |
| | 20% TBSA) | | |
| RC45 | Lacerations: (open wounds | 942.30 | Unadjusted |
| | without fractures)/Severe | | |
| | Burns (2nd degree >20- | | |
| | 44% TBSA*) | | |
| RC46 | Extremity fracture/Mild | 812.31 | Unadjusted |
| | Burns (2nd degree 6- | | |
| | 16% TBSA) | | |
| RC47 | Extremity fracture/Moderate | 942.00 | Unadjusted |
| | Burns (2nd degree >16- | | |
| | 20% TBSA) | | |
| RC48 | Extremity fracture/Severe | 942.30 | Unadjusted |
| | Burns (2nd degree >20- | | |
| | 44% TBSA*) | | |
| RC49 | Thoracic/abdominal tissue | 860.5 | Unadjusted |
| | trauma/Mild Burns (2nd | | |
| | degree 6-16% TBSA) | | |
| RC50 | Thoracic/abdominal tissue | 860.5 | Unadjusted |
| | trauma/Moderate Burns (2nd | | |
| | degree >16-20% TBSA) | | |
| RC51 | Thoracic/abdominal tissue | 860.5 | Unadjusted |
| | trauma/Severe Burns (2nd | | |
| | degree >20-44% TBSA*) | | |

Table 8 shows the mortality model for the radiological and nuclear patient codes.

TABLE 8
Patient Codes for Radiological and Nuclear Injuries

| | | | Treated |
|---|---|---|---|
| | | | Death |
| Patient Code | | Description | Probability |
| | RC01 | Mild Radiation Injury (0.7-1.25 Gy) | 0.00 |
| | RC02 | Moderate Radiation Injury (>1.25-3.0 Gy) | 0.06 |
| | RC03 | Severe Radiation Injury (>3.0-6.0 Gy) | 0.46 |
| | RC06 | Lacerations: (open wounds without fractures) | 0.46 |
| | | with Radiation Injury (>3.0-6.0 Gy) | |
| | RC10 | Head trauma: (cerebral concussion, w/or | 0.00 |
| | | w/o fracture) with Radiation Injury | |
| | | (0.7-1.2 . . . | |
| | RC11 | Head trauma: (cerebral concussion, w/or | 0.06 |
| | | w/o fracture) with Radiation Injury | |
| | | (>1.25-3 . . . | |
| | RC12 | Head trauma: (cerebral concussion, w/or | 0.46 |
| | | w/o fracture) with Radiation Injury | |
| | | (>3.0-6 . . . | |
| | RC15 | Extremity trauma open: (fracture humerus) | 0.46 |
| | | with Radiation Injury (. . . | |
| | RC19 | Thoracic fracture (wound thorax associated | 0.00 |
| | | with rib fractures) with Radiation Injury (. . . | |
| | RC20 | Thoracic fracture (wound thorax associated | 0.06 |
| | | with rib fractures) with Radiation Injury (. . . | |
| | RC21 | Thoracic fracture (wound thorax associated | 0.46 |
| | | with rib fractures) with Radiation Injury (. . . | |
| | RC27 | Mild Burns (2nd degree 6-16% TBSA) with | 0.46 |
| | | Radiation Injury (>3.0-6.0 Gy) | |
| » | RC52 | Expectant Injury | 1.00 |

Like other casualty types, casualties resulting from a radiological and nuclear attack can have an estimated time of death specified in the PSG. Casualties that use the radiological and nuclear mortality model will only model mortality when an initial estimated time of death is specified. This is because key treatment of radiological and nuclear injuries does not change the estimated time of death, but it can prevent death from occurring.

Key treatment occurs for casualties using the radiological and nuclear mortality model when treatment is performed at an MTF with Role 2 Enhanced (2E) or higher care. At this point, a random draw is compared to the casualty's treated death probability (Table 9) to see if death was prevented. If so, the future death event is removed. If not, the future death event remains unchanged. Error! Reference source not found. 12 shows the process for updating radiological/nuclear mortality based on treatment.

Although, DTRA has the capability to project CBRN casualty types and number. Currently, there is no method to produce 1) CBRN casualty distributions, 2) CBRN role 3 hospital bed, operating room, and ICU requirements, 3) CBRN theater blood requirements, 4) CBRN medical consumable and equipment requirements or 5) CBRN theater-wide medical laydown course of action analyses, transportation requirements evaluation, or projection of CBRN patient condition clinical outcomes. Therefore, the current improvements to MPTk and JMPT provide the added capability to medical planners, and allow them to assess the adequacy of their medical logistic plans based on different CBRN casualty distributions, and theater-wide medical laydown. The present invention also assist the medical planner to implement the best medical logistic plan by providing them with different medical requirements for preparation, including but not limited to requirements for hospital bed, operating room, and ICU, CBRN theater blood products, CBRN medical consumable and equipment requirements etc.

### Example 1: Generating a Medical Logistic Plan for an Operational Scenario Involving a Chemical, Biological, Radiological, or Nuclear Event

To generate a medical logistic plan for an operational scenario involving a CRBN event. The user must first selecting an operational scenario in MPTk based on the type of operation, the geographic location of the operation, and the duration of the operation. The user then creates a medical logistic plan in MPTk for the operational scenario by setting a plurality of parameters of said medical logistic plan. These parameters may include but not limited to location of one or more point of injures (POI), the locations and parameters of one or more medical treatment facility (MTF); transportation parameters; service discipline; location of the CBRN decontamination center, and CBRN treatment available at each MTF; and parameters of one or more casualty generation events. MTF may include Self/buddy Aid (SBA), First responder; battalion aid station (BAS), or shock trauma platoon (STP)/Forward Resuscitative Surgical System (FRSS). The MTFs are also categorized based on their role of care, which depends on the function of the MTF and type of medical care that is available to a casualty at this MTF. Because CREstT tool in MPTk does not generate CBRN casualties. The user need to reach back and requests estimates of CBRN casualties from the Defense Thread Reduction Agency (DTRA) based on the operational scenario, the medical logistic plan and the type of CBRN event selected. DTRA uses JEM to provide estimates of CBRN casualties. JEM takes into consideration the impacts of weather, terrain, and material interactions and generate CBRN casualties estimates including but not limited to patient conditions, effects for the number of casualties, the agent involved in the CBRN attack, type of attack and severity level. The user then incorporating these estimates about CBRN casualties from DTRA into MPTk and perform a simulation of the operational scenario with the medical logistic plan. A patient stream is then generated with to conventional casualties, CBRN casualties and combination casualties. Depending on the PC code and probabilities associated with each PC code, the user is able to estimate medical requirements necessary to treat each patient from the patient stream at different MTFs. Results of each simulation may include but not limited to patient disposition, casualty flow with a medical network set up per said medical logistic plan, casualty accumulation at each MTF within the planned medical network, medical consumable type and quantities, transportation usage, personnel utilization, facility usage and equipment utilization. It will also include estimate for the cost, weight and volume of the medical supplies. The simulation can be performed entirely in MPTk or partially in JMPT. In order to run simulation in JMPT, the user needs to import patient steam generated by the CREstT tool of MPTk. Armed time-phase medical requirements provided by the medical logistic plan. Users can start implementing the medical plan by securing and transporting medical requirements based on the time-phased estimates produced by the present invention. The medical requirements may include but not limited to operating rooms (OR), intensive care unit (ICU), ward beds, evacuations capabilities, Critical Care Air Transport Team (CCATT) capabilities, Class VIII consumable supplies, equipment; and medical personnel.

### Example 2. Assessing Medical Logistic Plan and Projecting Patient's Clinical Outcomes from the Point of Injury (POI) Through Evacuation from Theater

The present invention can also be used to generate medical laydowns for a medical treatment network within a theater, including their spatial/geographic arrangements, and assess the adequacy of a medical logistic plan.

To assess the adequacy of a medical logistic plan, a simulation is run according to example 1. The user can then calculate the died-of-wound probabilities of a patient stream for the simulated operational scenario involving a CBRN event. The present invention can estimate died-of-wound probability of each patient of the patient stream with conventional casualties; with CBRN casualties and calculate overall died-of wound probability for the patient stream of the simulation and report it to the user. The user can then adjust parameters of their medical logistic plan to run simulation again. By comparing the results of each simulation, the user will be able to assess the adequacy of their medical logistic plan based on died-of wound probability or needed medical requirements.

Instructions and illustrations for the operation of the present invention are provided in the Medical Planners' Toolkit User's Guide Version 1.5 and Joint Medical Planning Tool User's Manual Version 8.5, which are hereby incorporated by reference. Requests for these documents shall be referred to Naval Health Research Center (NHRC), Dept 161—Medical Modeling, Simulation, & Mission Support, 140 Sylvester Road, San Diego, Calif. 92016-3521.

What is claimed is:

1. A computer implemented method for planning medical logistic support of an operational scenario involving a CBRN agent, comprising:
a) selecting an operational scenario in MPTk;
b) creating a medical logistic plan in MPTk for said operational scenario by setting a plurality of parameters of said medical logistic plan;
c) requesting estimates of CBRN casualties from the Defense Thread Reduction Agency (DTRA) based on said operational scenario, said medical logistic plan and a selected CBRN type;
d) incorporating estimates of chemical casualties from DTRA into MPTk;
e) performing a simulation of said operational scenario using said parameters of said medical logistic plan to generate a patient stream with conventional casualties, CBRN casualties and combination casualties;
f) generating medical requirements necessary to treat said patient stream; and
g) implementing said medical logistic plan using medical requirements generated in step (f).

2. The method of claim 1, wherein step (a) further comprising
a) selecting the type of said operational scenario;
b) setting geographic location of said operational scenario; and
c) defining the duration of said operational scenario.

3. The method of claim 1, wherein step (b) further comprising:
a) identifying location of one or more point of injures (POI);
b) setting locations and parameters of one or more medical treatment facility (MTF);
c) defining transportation parameters;
d) defining service discipline;
e) defining CBRN Decontamination and treatment parameters; and
e) defining parameters of one or more casualty generation events.

4. The method of claim 3, wherein step (b) further comprising:
a) selecting a location for each of said MTF; and
b) defining the type and parameters of each of said MTF.

5 The method of claim 4, wherein said type of MTF comprising:
a) Self/buddy Aid (SBA);
b) First responder;
c) battalion aid station (BAS);
d) shock trauma platoon (STP)/Forward Resuscitative Surgical System (FRSS), or
e) CBRN decontamination center.

6. The method of claim 1, wherein said step (c) further comprising
a) providing DTRA information about said operational scenario involving a CBRN agent; and
b) receiving from DTRA time-phased estimates of CBRN casualties based on said operational scenario and said medical logistic plan, wherein said estimates of CBRN casualties include patient conditions, effects for the number of casualties, the agent involved in the CBRN attack, type of attack and severity level.

7. The method of claim 1, wherein results of said simulation comprising:
a) patient disposition;
b) casualty flow with a medical network set up per said medical logistic plan;
c) casualty accumulation at each MTF of said medical network;
d) medical consumable quantities;
e) transportation usage;
f) personnel utilization;
g) facility usage;
h) equipment utilization; or
h) a combined thereof.

8. The method of claim 1, wherein said medical requirements are generated by MPTk or JMPT.

9. The method of claim 8, wherein said medical requirements are selected from group consisting of:
a) operating rooms (OR);
b) intensive care unit (ICU);
c) ward beds;
d) evacuations capabilities;
e) Critical Care Air Transport Team (CCATT) capabilities;
f) Class VIII consumable supplies;
g) equipment;
h) personnel; or
h) a combination thereof.

10. The method of claim 9, wherein said medical requirements of claim 8 include estimates of cost, weight, and volume of said supply.

11. The method of claim 9, wherein said medical requirements is generated daily for each MTF of said medical network.

12. A computer implemented method for estimating died-of-wound probabilities of a patient stream for a simulated operational scenario involving a CBRN event, comprising:
a) selecting an operational scenario for a simulation in MPTk;
b) providing a series of interactive displays containing inquires for inputs by a user of a plurality of parameters concerning said operational scenario;
c) requesting estimates of CBRN casualties from the Defense Thread Reduction Agency (DTRA) based on said operational scenario, based on said medical logistic plan and a selected CBRN type;
d) incorporating estimates of CBRN casualties from DTRA into MPTk;
e) performing a simulation of said operational scenario using said parameters of said medical logistic plan to generate a patient stream;
f) estimating died-of-wound probability of each patient of said patient streams with conventional casualties;
e) estimating died-of-wound probability of each patient of said patient streams with CBRN casualties;
f) calculating overall died-of-wound probability for each patient of said simulation;
g) calculating overall died-of wound probability for said patient stream of said simulation; and
h) reporting said die-of wound probabilities to said user.

13. A computer implemented method for generating a patient steam for a simulated operational scenario with a CBRN event, comprising
a) selecting an operational scenario for simulation;
b) providing a series of interactive displays containing inquires for inputs by a user of a plurality of parameters for said operational scenario;
c) selecting one or more conventional casualty generating events;
d) importing estimates of CBRN casualties of said CBRN event;
e) setting parameters of each of said casualty generating events;
e) generating a patient stream for each said casualty generating events using a patient stream generator;
f) assigning a patient condition code to each patient of said patient steam; and
g) recording patient information for each patient of said patient stream.

14. The method of claim 13, wherein said parameters of said casualty event is comprising:
a) type of said casualty generator;
b) patient casualty occurrence frequencies (PCOF);
c) arrival time of said casualty generating event;
d) duration of said casualty generating event;
e) Patient Condition distribution, and
f) a point of injury (POI) and probabilities of injuries associated with said POI; wherein probabilities for all POI=1.

14. The method of claim 13, wherein said casualty generator comprising:
a) a single rate casualty generator;
b) a multiple rate patient stream generator; or
c) a mass casualty event patient steam generator.

15. The method of claim 13, wherein said type of casualty comprising:
a) wound in action (WIA);
b) disease;
c) Nonbattle Injury (NBI); or
d) Combat Stress (CS).

16. The method of claim 13, wherein said patient information comprising:
a) KIA determination;
b) Patient condition;
c) POI; and
d) Time of injury.
